(19) United States
(12) Patent Application Publication
(10) Pub. No.: US 2021/0125091 A1
(43) Pub. Date: Apr. 29, 2021

(54) PREDICTIVE DATA ANALYSIS WITH CATEGORICAL INPUT DATA

(71) Applicant: Optum Services (Ireland) Limited, Dublin (IE)

(72) Inventors: Dong Fang, Dublin (IE); Peter Cogan, Dublin (IE)

(21) Appl. No.: 16/661,053

(22) Filed: Oct. 23, 2019

(51) Int. Cl. G06N 5/04; G06N 20/10

(52) U.S. Cl. CPC G06N 5/046; G06N 20/10

(57) ABSTRACT

There is a need for more effective and efficient predictive data analysis solutions that utilize categorical input data objects. This need can be addressed by, for example, solutions for performing predictive inference using a categorical inference machine learning engine. In one example, a method includes receiving categorical input data objects, generating, based on each particular categorical input data object and using embedding layers, embedded feature representations for the particular categorical input data object; generating, based on each particular embedded feature representation and using initial capsule layers; initial instantiation parameters for the corresponding categorical data object; generating, based on each initial instantiation parameter and using subsequent capsule layers, inferred instantiation parameters for categorical input data objects; and generating predictions based at least in part on the inferred instantiation parameters.

### BACKGROUND

Various embodiments of the present invention address technical challenges related to performing predictive data analysis. Existing predictive data analysis solutions are ill-suited to efficiently and reliably perform predictive data analysis using categorical input data. Various embodiments of the present address the shortcomings of the noted feedback mining systems and disclose various techniques for efficiently and reliably performing predictive data analysis using categorical input data.

### BRIEF SUMMARY

In general, embodiments of the present invention provide methods, apparatus, systems, computing devices, computing entities, and/or the like for performing predictive data analysis using categorical input data. Certain embodiments utilize systems, methods, and computer program products that perform machine learning predictive inferences using categorical input data by utilizing one or more of initial capsule layers, spatial fully-connected (FC) layers. time-distributed layers, localized convolutional layers, value designations regimes for categorical data objects, regime-specific feature processing layers, regime-specific prediction layers, error designations for training data objects, error-designation-specific loss models, etc.

In accordance with one aspect, a method is provided. In one embodiment, the method comprises receiving one or more categorical input data objects, wherein each of the categorical input data objects is associated with one or more categorical feature values; generating, using one or more embedding layers of a categorical inference machine learning engine and based at least in part on each of the categorical input data objects, one or more embedded feature representations for the corresponding categorical input data object; for each embedded feature representation associated with the corresponding categorical input data object, generating, using one or more initial capsule layers of the categorical inference machine learning engine and based at least in part on the corresponding embedded feature representation, one or more initial instantiation parameters indicating an extracted occurrence property of the corresponding embedded feature representation with respect to the corresponding categorical input data object; generating, using one or more subsequent capsule layers and based at least in part on each initial instantiation parameter, one or more inferred instantiation parameters for the corresponding categorical input data object, wherein each inferred instantiation parameter for the corresponding categorical input data object indicates an inferred occurrence property of a corresponding inferred attribute with respect to the corresponding categorical input data object; and generating one or more predictions based at least in part on each of the one or more inferred instantiation parameters.

In accordance with another aspect, a computer program product is provided. The computer program product may comprise at least one computer-readable storage medium having computer-readable program code portions stored therein, the computer-readable program code portions comprising executable portions configured to receive one or more categorical input data objects, wherein each of the categorical input data objects is associated with one or more categorical feature values; generate, using one or more embedding layers of a categorical inference machine learning engine and based at least in part on each of the categorical input data objects, one or more embedded feature representations for the corresponding categorical input data object; for each embedded feature representation associated with the corresponding categorical input data object, generate, using one or more initial capsule layers of the categorical inference machine learning engine and based at least in part on the corresponding embedded feature representation, one or more initial instantiation parameters indicating an extracted occurrence property of the corresponding embedded feature representation with respect to the corresponding categorical input data object; generate, using one or more subsequent capsule layers and based at least in part on each initial instantiation parameter, one or more inferred instantiation parameters for the corresponding categorical input data object, wherein each inferred instantiation parameter for the corresponding categorical input data object indicates an inferred occurrence property of a corresponding inferred attribute with respect to the corresponding categorical input data object; and generate one or more predictions based at least in part on each of the one or more inferred instantiation parameters.

In accordance with yet another aspect, an apparatus comprising at least one processor and at least one memory including computer program code is provided. In one embodiment, the at least one memory and the computer program code may be configured to, with the processor, cause the apparatus to receive one or more categorical input data objects, wherein each of the categorical input data objects is associated with one or more categorical feature values; generate, using one or more embedding layers of a categorical inference machine learning engine and based at least in part on each of the categorical input data objects, one or more embedded feature representations for the corresponding categorical input data object; for each embedded feature representation associated with the corresponding categorical input data object, generate, using one or more initial capsule layers of the categorical inference machine learning engine and based at least in part on the corresponding embedded feature representation, one or more initial instantiation parameters indicating an extracted occurrence property of the corresponding embedded feature representation with respect to the corresponding categorical input data object; generate, using one or more subsequent capsule layers and based at least in part on each initial instantiation parameter, one or more inferred instantiation parameters for the corresponding categorical input data object, wherein each inferred instantiation parameter for the corresponding categorical input data object indicates an inferred occurrence property of a corresponding inferred attribute with respect to the corresponding categorical input data object; and generate one or more predictions based at least in part on each of the one or more inferred instantiation parameters.

### BRIEF DESCRIPTION OF THE DRAWINGS

Having thus described the invention in general terms, reference will now be made to the accompanying drawings, which are not necessarily drawn to scale, and wherein:

FIG. 1 provides an exemplary overview of an architecture that can be used to practice embodiments of the present invention.

FIG. 2 provides an example categorical inference computing entity in accordance with some embodiments discussed herein.

FIG. 3 provides an example client computing entity in accordance with some embodiments discussed herein.

FIG. 4 is a data flow diagram of an example process for performing general categorical predictive inference based at least in part on categorical data objects in accordance with some embodiments discussed herein.

FIG. 5 is a data flow diagram of an example process for generating embedded feature representations for categorical data objects in accordance with some embodiments discussed herein.

FIG. 6 is a data flow diagram of an example process for generating initial instantiation parameters for categorical data objects in accordance with some embodiments discussed herein.

FIG. 7 is a data flow diagram of an example process for performing regime-specific categorical predictive inference based at least in part on categorical data objects in accordance with some embodiments discussed herein.

FIG. 8 is a flowchart diagram of an example process for training a categorical inference machine learning engine in accordance with some embodiments discussed herein.

### DETAILED DESCRIPTION

Various embodiments of the present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout. Moreover, while certain embodiments of the present invention are described with reference to predictive data analysis, one of ordinary skill in the art will recognize that the disclosed concepts can be used to perform other types of data analysis.

### I. OVERVIEW

Discussed herein methods, apparatus, systems, computing devices, computing entities, and/or the like for performing predictive data analysis using categorical input data. As will be recognized, however, at least some of the disclosed concepts (e.g., concepts related to error-designation-specific loss models) can be used to perform any type of data analysis and/or predictive data analysis using non-categorical types of input data.

Various embodiments of the present invention improve efficiency and effectiveness of predictive data analysis using categorical input data. Categorical input data includes feature values that are selected from a range of discrete categories rather than a numeric range. Because many state-of-the-art machine learning models are designed with numeric input data in mind, predictive data analysis using categorical input data has lagged behind many other areas of predictive data analysis. For example, many convolutional models and capsule-based models (e.g., the CapsNet model) have not been heavily utilized in relation to categorical input data because of the non-numeric semantics of such input data. In rare instances where complex numeric models have been used to process categorical data, naïve attempts to translate categorical data to numeric equivalents that fail to learn from semantic structures of categorical data have rendered such solutions ineffective and unreliable. As a result, existing predictive data analysis solutions that use categorical input data are largely inefficient to train and unreliable in performing effective predictive inferences even when trained.

Various aspects of the present invention address the technical challenges associated with efficiency and reliability of existing categorical predictive inference solutions. For example, according to one aspect, instantiation parameters for categorical data are generated based at least in part on embedded representations of such categorical data and by a set of spatial FC layers followed by a 1-dimensional localized convolutional layer. Such instantiation parameters can in turn be used by sophisticated numeric machine learning models (e.g., by a primary capsule layer in the CapsNet model) to generate feature models of categorical input data that include strong predictive signals. As another example, according to another aspect of the present invention, categorical data can be split into various distinct regimes (e.g., value-based regimes), where at least a portion of the predictive inferences using each of the various regimes is performed independently from other regimes and using separate parameters in order to capture semantic information about diversity of predictive signals associated with the underlying domains providing categorical input data. As a further example, according to yet another aspect of the present invention, categorical inference machine learning engines can be trained using hybrid loss models utilized for various error designations associated with the categorical input data, which in turn facilitates performing better parameter updating that takes into account various loss profiles associated with varying segments of data, thus increasing training efficiency and training effectiveness of predictive data analysis models utilizing categorical input data.

By utilizing those and other aspects, various embodiments of the present invention address various technical shortcomings of existing categorical predictive inference solutions, address various technical challenges related to performing predictive data analysis using categorical input data, and make important technical contributions to improving efficiency and effectiveness of performing predictive data analysis using categorical input data.

### II. COMPUTER PROGRAM PRODUCTS, METHODS, AND COMPUTING ENTITIES

Embodiments of the present invention may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatus, systems, computing devices, computing entities, and/or the like. As such, embodiments of the present invention may take the form of an apparatus, system, computing device, computing entity, and/or the like executing instructions stored on a computer-readable storage medium to perform certain steps or operations. Thus, embodiments of the present invention may also take the form of an entirely hardware embodiment, an entirely computer program product embodiment, and/or an embodiment that comprises combination of computer program products and hardware performing certain steps or operations. Embodiments of the present invention are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### III. EXEMPLARY SYSTEM ARCHITECTURE

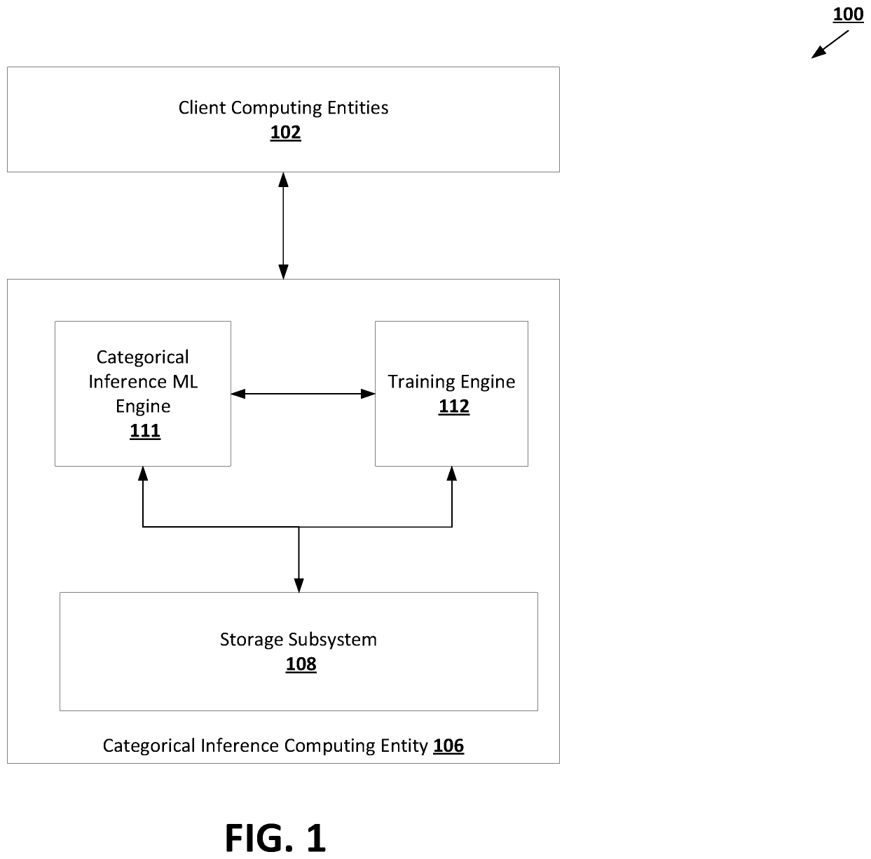

FIG. 1 is a schematic diagram of an example architecture 100 for performing predictive data analysis using categorical input data. The architecture 100 includes one or more client computing entities 102 and a categorical inference computing entity 106. The categorical inference computing entity 106 may be configured to communicate with at least one of the client computing entities 102 over a communication network (not shown). The communication network may include any wired or wireless communication network including, for example, a wired or wireless local area network (LAN), personal area network (PAN), metropolitan area network (MAN), wide area network (WAN), or the like, as well as any hardware, software and/or firmware required to implement it (such as, e.g., network routers, and/or the like).

A client computing entity 102 may be configured to provide predictive requests to the categorical inference computing entity 106 and receive corresponding predictive outputs form the categorical inference computing entity 106. The predictive requests from the client computing entity 102 may at least in part require performing predictive data analysis using categorical input data. For example, a client computing entity 102 may provide information about various medical claims to the categorical inference computing entity 106 and in response request predictions about which of the various medical claims should be flagged for further review and/or for automatic price adjustment. As another example, a client computing entity 102 may provide information about various medical claims to the categorical inference computing entity 106 and in response request predictions about suitable values for each of the various medical claims. As a further example, a client computing entity 102 may provide information about various medical claims to the categorical inference computing entity 106 and in response request predictions about quality metrics of the various medical claims.

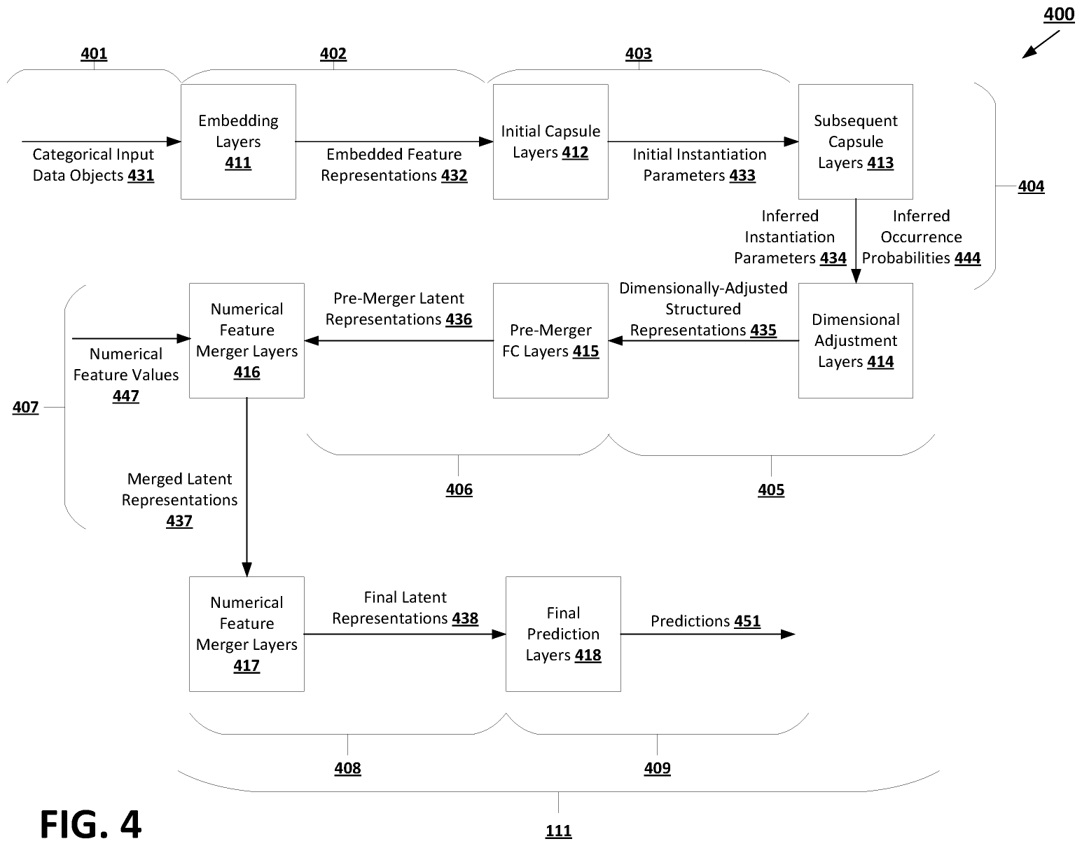

The categorical inference computing entity 106 is configured to perform predictive inferences using categorical input data in order to generate predictions based at least in part on the categorical input data. To do so, the categorical inference computing entity 106 utilizes a categorical inference machine learning engine 111 trained by a training engine 112. Various operations of the categorical inference machine learning engine 111 and the training engine 112 are described below with reference to FIGS. 4-8. Moreover, the categorical inference computing entity 106 includes a storage subsystem 108 configured to store at least one of hyper-parameter data associated with the categorical inference machine learning engine 111, hyper-parameter data associated with the training engine 112, categorical input data utilized by the categorical inference machine learning engine 111, training data utilized by the training engine 112, configuration data for the categorical inference computing entity 106, etc.

The storage subsystem 108 may include one or more storage units, such as multiple distributed storage units that are connected through a computer network. Each storage unit in the storage subsystem 108 may store at least one of one or more data assets and/or one or more data about the computed properties of one or more data assets. Moreover, each storage unit in the storage subsystem 108 may include one or more non-volatile storage or memory media including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

### Exemplary Categorical Inference Computing Entity

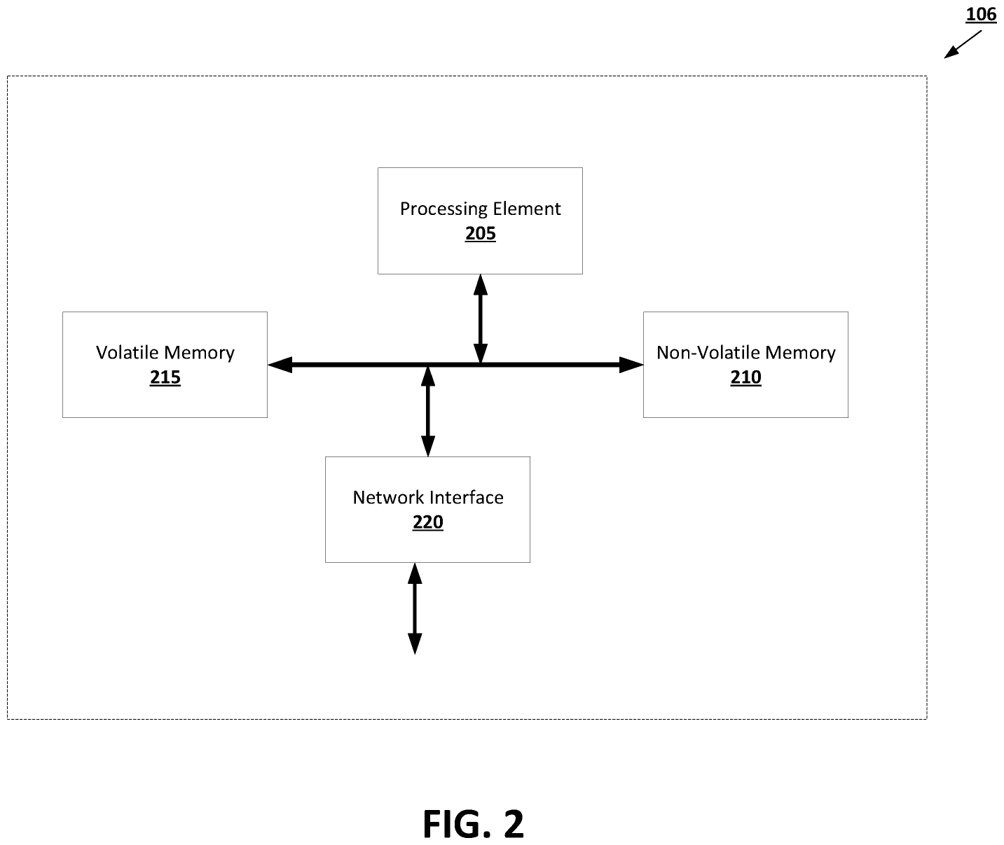

FIG. 2 provides a schematic of a categorical inference computing entity 106 according to one embodiment of the present invention. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

As indicated, in one embodiment, the categorical inference computing entity 106 may also include one or more communications interfaces 220 for communicating with various computing entities, such as by communicating data, content, information, and/or similar terms used herein interchangeably that can be transmitted, received, operated on, processed, displayed, stored, and/or the like.

As shown in FIG. 2, in one embodiment, the categorical inference computing entity 106 may include or be in communication with one or more processing elements 205 (also referred to as processors, processing circuitry, and/or similar terms used herein interchangeably) that communicate with other elements within the categorical inference computing entity 106 via a bus, for example. As will be understood, the processing element 205 may be embodied in a number of different ways. For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing elements 205 may be embodied as integrated circuits, application specific integrated circuits (ASICs), field programmable gate arrays (FPGAs), programmable logic arrays (PLAs), hardware accelerators, other circuitry, and/or the like. As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present invention when configured accordingly.

In one embodiment, the categorical inference computing entity 106 may further include or be in communication with non-volatile media (also referred to as non-volatile storage, memory, memory storage, memory circuitry and/or similar terms used herein interchangeably). In one embodiment, the non-volatile storage or memory may include one or more non-volatile storage or memory media 210, including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

In one embodiment, the categorical inference computing entity 106 may further include or be in communication with volatile media (also referred to as volatile storage, memory, memory storage, memory circuitry and/or similar terms used herein interchangeably). In one embodiment, the volatile storage or memory may also include one or more volatile storage or memory media 215, including but not limited to RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain aspects of the operation of the categorical inference computing entity 106 with the assistance of the processing element 205 and operating system.

As indicated, in one embodiment, the categorical inference computing entity 106 may also include one or more communications interfaces 220 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the categorical inference computing entity 106 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the categorical inference computing entity 106 may include or be in communication with one or more input elements, such as a keyboard input, a mouse input, a touch screen/display input, motion input, movement input, audio input, pointing device input, joystick input, keypad input, and/or the like. The categorical inference computing entity 106 may also include or be in communication with one or more output elements (not shown), such as audio output, video output, screen/display output, motion output, movement output, and/or the like.

### Exemplary Client Computing Entity

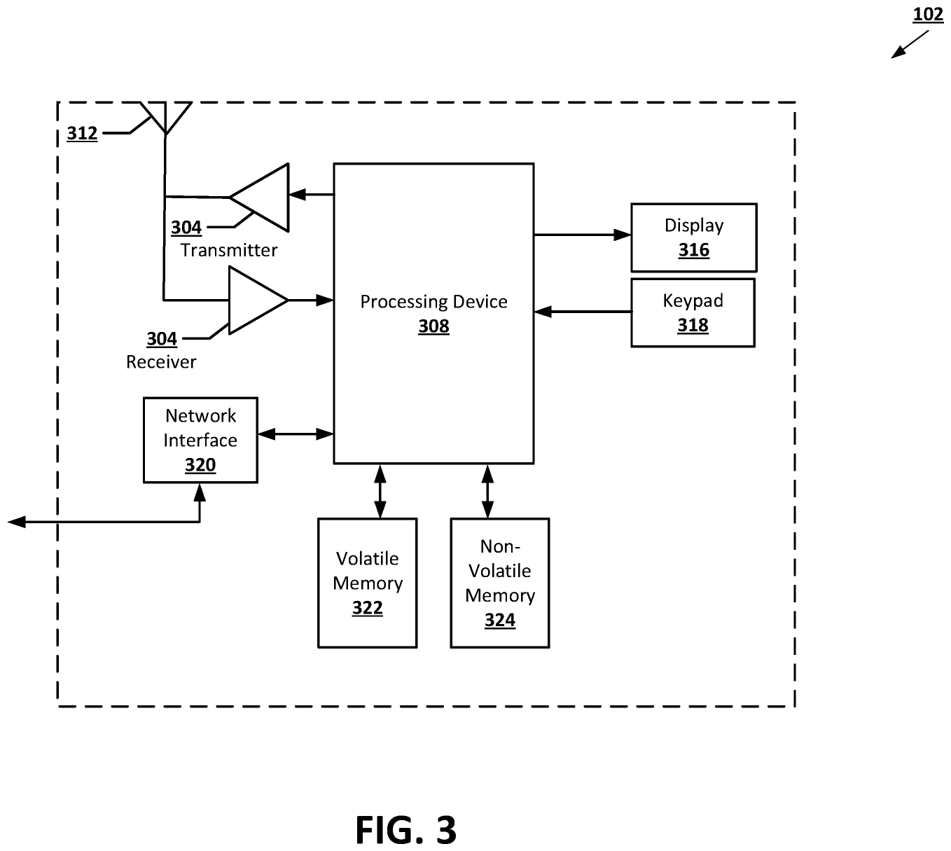

FIG. 3 provides an illustrative schematic representative of a client computing entity 102 that can be used in conjunction with embodiments of the present invention. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Client computing entities 102 can be operated by various parties. As shown in FIG. 3, the client computing entity 102 can include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly.

The signals provided to and received from the transmitter 304 and the receiver 306, correspondingly, may include signaling information/data in accordance with air interface standards of applicable wireless systems. In this regard, the client computing entity 102 may be capable of operating with one or more air interface standards, communication protocols, modulation types, and access types. More particularly, the client computing entity 102 may operate in accordance with any of a number of wireless communication standards and protocols, such as those described above with regard to the categorical inference computing entity 106. In a particular embodiment, the client computing entity 102 may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the client computing entity 102 may operate in accordance with multiple wired communication standards and protocols, such as those described above with regard to the categorical inference computing entity 106 via a network interface 320.

Via these communication standards and protocols, the client computing entity 102 can communicate with various other entities using concepts such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The client computing entity 102 can also download changes, add-ons, and updates, for instance, to its firmware, software (e.g., including executable instructions, applications, program modules), and operating system.

According to one embodiment, the client computing entity 102 may include location determining aspects, devices, modules, functionalities, and/or similar words used herein interchangeably. For example, the client computing entity 102 may include outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the client computing entity's 102 position in connection with a variety of other systems, including cellular towers, Wi-Fi access points, and/or the like. Similarly, the client computing entity 102 may include indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The client computing entity 102 may also comprise a user interface (that can include a display 316 coupled to a processing element 308) and/or a user input interface (coupled to a processing element 308). For example, the user interface may be a user application, browser, user interface, and/or similar words used herein interchangeably executing on and/or accessible via the client computing entity 102 to interact with and/or cause display of information/data from the categorical inference computing entity 106, as described herein. The user input interface can comprise any of a number of devices or interfaces allowing the client computing entity 102 to receive data, such as a keypad 318 (hard or soft), a touch display, voice/speech or motion interfaces, or other input device. In embodiments including a keypad 318, the keypad 318 can include (or cause display of) the conventional numeric (0-9) and related keys (#, *), and other keys used for operating the client computing entity 102 and may include a full set of alphabetic keys or set of keys that may be activated to provide a full set of alphanumeric keys. In addition to providing input, the user input interface can be used, for example, to activate or deactivate certain functions, such as screen savers and/or sleep modes.

The client computing entity 102 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the client computing entity 102. As indicated, this may include a user application that is resident on the entity or accessible through a browser or other user interface for communicating with the categorical inference computing entity 106 and/or various other computing entities.

In another embodiment, the client computing entity 102 may include one or more components or functionality that are the same or similar to those of the categorical inference computing entity 106, as described in greater detail above. As will be recognized, these architectures and descriptions are provided for exemplary purposes only and are not limiting to the various embodiments.

In various embodiments, the client computing entity 102 may be embodied as an artificial intelligence (AI) computing entity, such as an Amazon Echo, Amazon Echo Dot, Amazon Show, Google Home, and/or the like. Accordingly, the client computing entity 102 may be configured to provide and/or receive information/data from a user via an input/output mechanism, such as a display, a camera, a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

### IV. EXEMPLARY SYSTEM OPERATIONS

Various embodiments of the present invention improve efficiency and effectiveness of predictive data analysis using categorical input data. Categorical input data includes feature values that are selected from a range of discrete categories rather than a numeric range. Because many state-of-the-art machine learning models are designed with numeric input data in mind, predictive data analysis using categorical input data has lagged behind many other areas of predictive data analysis. For example, many convolutional models and capsule-based models (e.g., the CapsNet model) have not been heavily utilized in relation to categorical input data because of the non-numeric semantics of such input data. In rare instances where complex numeric models have been used to process categorical data, naïve attempts to translate categorical data to numeric equivalents that fail to learn from semantic structures of categorical data have rendered such solutions ineffective and unreliable. As a result, existing predictive data analysis solutions that use categorical input data are largely inefficient to train and unreliable in performing effective predictive inferences even when trained.

Various aspects of the present invention address the technical challenges associated with efficiency and reliability of existing categorical predictive inference solutions. For example, according to one aspect, instantiation parameters for categorical data are generated based at least in part on embedded representations of such categorical data and by a set of spatial FC layers followed by a 1-dimensional localized convolutional layer. Such instantiation parameters can in turn be used by sophisticated numeric machine learning models (e.g., by a primary capsule layer in the CapsNet model) to generate feature models of categorical input data that include strong predictive signals. As another example, according to another aspect of the present invention, categorical data can be split into various distinct regimes (e.g., value-based regimes), where at least a portion of the predictive inferences using each of the various regimes is performed independently from other regimes and using separate parameters in order to capture semantic information about diversity of predictive signals associated with the underlying domains providing categorical input data. As a further example, according to yet another aspect of the present invention, categorical inference machine learning engines can be trained using hybrid loss models utilized for various error designations associated with the categorical input data, which in turn facilitates performing better parameter updating that takes into account various loss profiles associated with varying segments of data, thus increasing training efficiency and training effectiveness of predictive data analysis models utilizing categorical input data.

By utilizing those and other aspects, various embodiments of the present invention address various technical shortcomings of existing categorical predictive inference solutions, address various technical challenges related to performing predictive data analysis using categorical input data, and make important technical contributions to improving efficiency and effectiveness of performing predictive data analysis using categorical input data.

A. General Categorical Predictive Inference

FIG. 4 is a data flow diagram of an example process 400 for performing a general (i.e., non-regime-based) predictive inference based at least in part on categorical input data objects 431. Via the various steps/operations depicted in process 400, the categorical inference machine learning engine 111 of the categorical inference computing entity 106 can perform effective and efficient predictive inferences based at least in part on a general stream of categorical input data objects 431 in order to generate reliable and effective predictions 451.

The process depicted in process 400 begins at step/operation 401 when the embedding layers 411 of the categorical inference machine learning engine 111 receive the categorical input data objects 431. In some embodiments, a categorical input data object is a data object that includes at least one categorical feature value, where a categorical feature value is a value that indicates association of the categorical input data object with a selected category of a plurality of discrete candidate categories. Each categorical input data object 431 may correspond to a predictive entity and include one or more categorical feature values, where each categorical feature value associated with a categorical input data object may in turn be associated with a categorical feature of one or more categorical features.

An example of a categorical input data object is a medical service event data object that includes categorical information about a medical service event predictive entity (e.g., a medical visitation event predictive entity, a medical operation event predictive entity, a drug purchase event predictive entity, etc.). Examples of categorical feature values for a medical service event data object may include location-identifying categorical feature values for a medical service predictive entity, medical-procedure-code-based categorical feature values for a medical service predictive entity, medical-diagnosis-code-based categorical feature values (e.g., medical-diagnosis-code-based categorical feature values characterized by a medical diagnoses classification system such as the Diagnosis-Related Group (DRG) system) for a medical service predictive entity, point-of-service-related categorical feature values for a medical service predictive entity, etc. In the discussed example, a particular location-identifying categorical feature value may be associated with a categorical feature that relates to a state identifier associated with a geographic region within which the corresponding medical service predictive entity is recorded to have occurred.

At step/operation 402, the embedding layers 411 of the categorical inference machine learning engine 111 process the categorical input data objects 431 to generate one or more embedded feature representations 432 for each categorical input data object 431 and provides the generated embedded feature representations 432 to initial capsule layers 412 of the categorical inference machine learning engine 111. In some embodiments, an embedded feature representation is a mapping of one or more categorical feature values to an n-dimensional space, where each feature dimension of the n feature dimensions may be characterized by a numeric range and where the dimension count n may be defined by one or more hyper-parameters of the categorical inference machine learning engine 111. In some embodiments, an embedded feature representation is a mapping of a numerical token (e.g., an integer token) generated based at least in part on one or more categorical features value to an n-dimensional space, where each feature dimension of the n feature dimensions may be characterized by a numeric range and where the dimension count n may be defined by one or more hyper-parameters of the categorical inference machine learning engine 111.

In some embodiments, to generate an embedded feature representation 432 based at least in part on a categorical feature value associated with a categorical input data object 431, the embedding layers 411 first tokenize the categorical feature value as an integer and then maps the tokenized categorical feature value to an n-dimensional space based at least in part on a look-up table, where at least some of the parameters defining the look-up table may be learned through at least one training procedure. In some embodiments, to generate an embedded feature representation 432 based at least in part on a categorical feature value associated with a categorical input data object 431, the embedding layers 411 perform one-hot encoding on the feature value. In general, any combination of one or more embedding techniques can be utilized to convert at least one categorical feature value into a corresponding embedded feature representation 432.

In some embodiments, the embedding layers 411 are configured to map categorical feature values associated with various distinct categorical features into embedded feature representations 432 of the same length and the same structure, e.g., vectors of length n where each value of the vector represents the same ordered set of embedded features across the various categorical feature values. In some embodiments, each embedded feature representation 432 has a shared embedding structure relative to the other embedded feature representations 432. In some embodiments, the embedding layers 411 are configured to map categorical feature values associated with distinct categorical features into embedded feature representations 432 having feature-specific representations. For example, categorical feature values having a first categorical feature type may be mapped to a n-dimensional space characterized by the d1-dn feature dimensions while categorical feature values having a second categorical feature type may be mapped to a n-dimensional space having dn+1−dn+m feature dimensions.

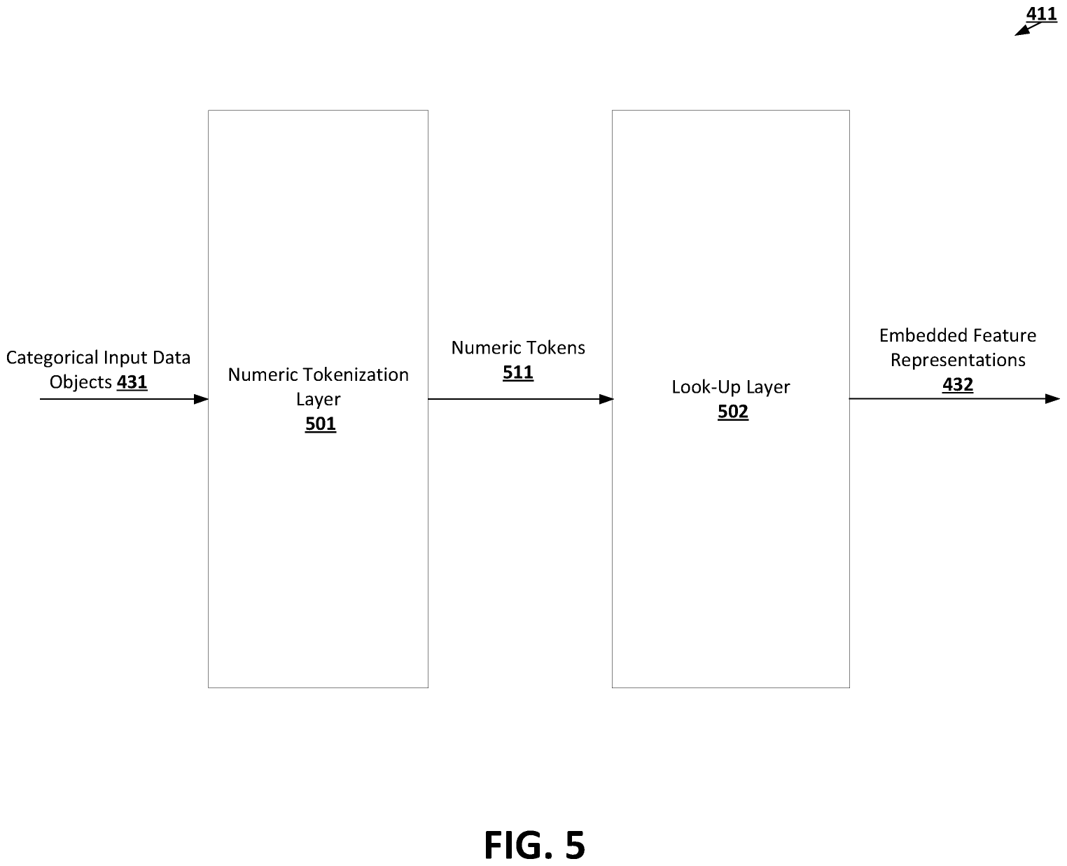

In some embodiments, step/operation 402 may be performed in accordance with the process depicted in FIG. 5, which is a data flow diagram of an example process for generating the embedded feature representations 432 for the categorical input data objects 431 using the embedding layers 411. As depicted in FIG. 5, the embedding layers 411 include a numeric tokenization layer 501 that is configured to generate numeric tokens 511 corresponding to the categorical feature values associated with the categorical input data objects 431. For example, the numeric tokenization layer 501 may generate a numeric token 511 for each candidate state identifier value (e.g., may associate a state identifier value describing the state of Georgia to the number 21, a state identifier value describing the state of New York to 25, etc.). In some embodiments, the numeric tokenization layer 501 may convert categorical feature values to numeric tokens 511 based at least in part on one or more tokenization parameters, such as at least one of static tokenization parameters whose value is determinable prior to runtime, dynamic tokenization parameters whose value is determined at runtime, learned tokenization parameters determined using one or more training procedures, etc.

As further depicted in FIG. 5, the embedding layers 411 include a look-up layer 502 configured to map the numeric tokens 511 generated by the numeric tokenization layer 501 to embedded feature representations 432, e.g., embedded feature vectors having an n-dimensional structure. To map the numeric tokens 511 generated by the numeric tokenization layer 501 to the embedded feature representations 432, the embedding layers may utilize a look-up table configured to include mapping information for mapping numeric tokens 511 to corresponding n-dimensional feature spaces. In some embodiments, at least some of the parameters defining the look-up table may be learned through at least one training procedure.

Returning to FIG. 4, at step/operations 403, the initial capsule layers 412 of the categorical inference machine learning engine 111 process the embedded feature representations 432 to generate one or more instantiation parameters 433 for each embedded feature representation 432. In some embodiments, an initial instantiation parameter 433 for a corresponding embedded feature representation 432 that is in turn associated with a corresponding categorical input data object 431 describes an extracted occurrence property of the corresponding embedded feature representation 432 with respect to the corresponding embedded feature representation 432. For example, a particular initial instantiation parameter 433 may describe an orientation of the corresponding embedded feature representation 432 within a spatial space generated based at least in part on the corresponding categorical input data object 431. As another example, a particular initial instantiation parameter 433 may describe an intensity of occurrence of the corresponding embedded feature representation 432 with respect to the corresponding categorical input data object 431. As a further example, a particular initial instantiation parameter 433 may describe a predictive significance of the corresponding embedded feature representation 432 to making particular predictive inferences.

In some embodiments, the initial capsule layers 412 further generate an initial occurrence probability for an embedded feature representation. In some embodiments, an initial occurrence probability for a corresponding embedded feature representation 432 that is in turn associated with a corresponding categorical input data object 431 describes a probability of occurrence of the corresponding embedded feature representation 432 with respect to the corresponding categorical input data object 431. For example, a particular initial occurrence probability may describe a likelihood that the corresponding embedded feature representation 432 describes a property of the corresponding categorical input data object 431. The initial capsule layers 412 may provide the initial instantiation parameters 434 and/or the initial occurrence probabilities to subsequent capsule layers 403 of the categorical inference machine learning engine 111.

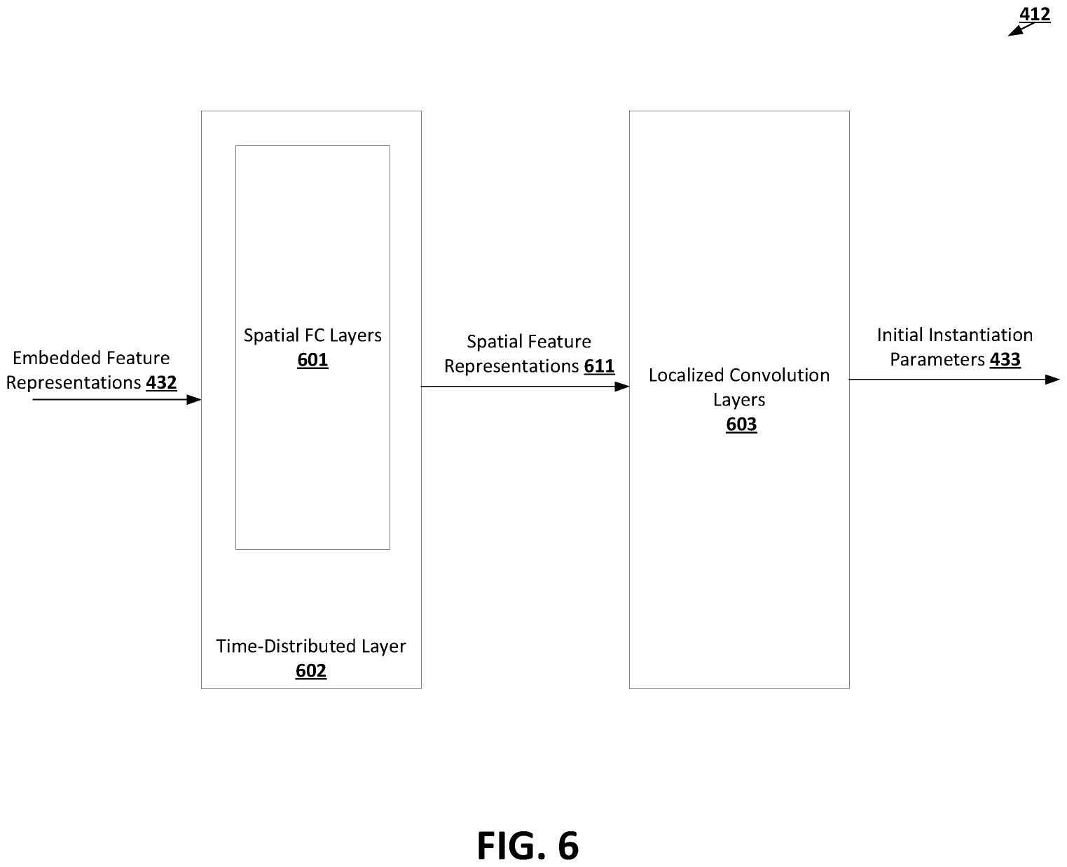

In some embodiments, step/operation 403 may be performed in accordance with the process depicted in FIG. 6, which is a data flow diagram of an example process for generating, by using the initial capsule layers 412, initial instantiation parameters 433 for embedded feature representation 432 with respect to categorical input data objects 431. As depicted in FIG. 6, the initial capsule layers 412 comprise spatial FC layers 601 which are wrapped by a time-distributed layer 602. The spatial FC layers 601 may be configured to process each embedded feature representation 432 that is associated with a categorical input data object 431 based at least in part on a relationship (e.g., a spatial relationship) between the embedded feature representation 432 and the categorical input data object 431 to generate a spatial feature representation 611 for the embedded feature representation 432. The spatial feature representation 611 for an embedded feature representation 432 may be determined at least in part by modeling the values defining embedded feature representation 432 into various spatial regions.

For example, the spatial FC layers 601 may be configured to process the embedded feature representation 432 based at least in part on information about other embedded feature representations 432 that are also associated with a corresponding categorical input data object 431 in order to generate the spatial feature representation 611 for the embedded feature representation 432. As another example, the spatial FC layers 601 may be configured to: (i) in a first set of spatial FC layers 601, apply a first set of parameters to each embedded feature representation 432 associated with a particular categorical input data object 431 in order to generate a set of first layer outputs; and (ii) in a second set of spatial FC layers 601, apply a second set of parameters to the set of first layer outputs to generate the spatial feature representation 611 for each embedded feature representation 432. In at least some of those embodiments, the fully-connected structure of the spatial FC layers 601 facilitates predictive inferences across various embedding feature representations 432 associated with the same categorical input data object 431.

In some embodiments, the spatial FC layers 601 are configured to share parameters across various categorical input data objects 431, e.g., across all of the categorical input data objects 431, across each portion of the categorical input data objects 431 that corresponds to the same predictive entity, across each portion of the categorical input data objects 431 that corresponds to a family of related predictive entities, etc. To do so, the spatial FC layers 601 may utilize the time-distributed layer 602 (e.g., the time-distributed layer in the Keras framework) as a wrapper layer for the spatial FC layers 601. In some embodiments, the time-distributed layer 602 is configured to generate spatial FC layers 601 corresponding to each categorical input data object 431 of the categorical input data objects 431 received in step/operation 401.

As further depicted in FIG. 6, the initial capsule layers 412 further comprise localized convolution layers 603 that are configured to process each spatial feature representation 611 for an embedded feature representation 432 in accordance with one or more feature extraction kernels to generate the initial instantiation parameters 433 for the embedded feature representation 432. A feature extraction kernel may be a computer-implemented routine configured to combine at least a portion of values (e.g., a region of values) in any particular spatial feature representation 611 to generate an initial instantiation parameter 433 corresponding to the particular spatial feature representation 611. For example, a feature extraction kernel may be configured to, from ten values in a particular spatial feature representation 611, apply a first parameter to a first value in the particular spatial feature representation 611, apply a second parameter to an eight value in the particular spatial feature representation 611, and combine the noted outputs to generate an initial instantiation parameter 433 corresponding to the particular spatial feature representation 611. As another example, given a particular spatial feature representation 611 defined using five spatial regions, a feature extraction kernel may use a first spatial region to determine an initial instantiation parameter. In some embodiments, the parameters associated with the feature extraction kernels may be determined using at least one training procedure.

Returning to FIG. 4, at step/operation 404, the subsequent capsule layers 413 of the categorical inference machine learning engine 111 process the initial instantiation parameters 433 for the embedded feature representations 432 (and optionally the initial feature probabilities for the embedded feature representations 432 if such values are generated by the initial capsule layers 412) to generate one or more inferred instantiation parameters 434 for each categorical input data object 431 and one or more inferred occurrence probabilities 444 for each categorical input data object 431. An inferred instantiation parameter 434 for a categorical input data object 431 may describe an inferred occurrence property of a corresponding inferred attribute with respect to the particular categorical input data object 431. An inferred occurrence probability 444 for a categorical input data object 431 may describe a predicted probability of occurrence of a corresponding inferred attribute with respect to the categorical input data object 431. The subsequent capsule layers 413 may provide the inferred instantiation parameters 434 and/or the inferred occurrence probabilities 444 to dimension-adjustment layers 414 of the categorical inference machine learning engine 111.

For example, a particular inferred instantiation parameter 434 may describe a predicted orientation of occurrence of a corresponding inferred attribute within a spatial space generated based at least in part on the corresponding categorical input data object 431. As another example, a particular inferred instantiation parameter 434 may describe a predicted intensity of occurrence of a corresponding inferred attribute with respect to the corresponding categorical input data object 431. As yet another example, a particular inferred instantiation parameter 434 may describe a predictive significance of the corresponding inferred attribute to making particular predictive inferences. As a further example, a particular inferred occurrence probability 444 may describe a likelihood that a particular categorical input data object 431 is associated with a corresponding inferred attribute.

In some embodiments, the range of inferred attributes characterizing the inferred instantiation parameters 434 and the inferred occurrence probabilities 444 may be determined based at least in part on a range of features whose values are determinable by particular capsules in a CapsNet machine learning architecture. For example, the range of inferred attributes characterizing the inferred instantiation parameters 434 and the inferred occurrence probabilities 444 may be determined based at least in part on a range of features whose values are determinable by particular capsules in a primary capsule layer of a CapsNet machine learning architecture. As another example, the range of inferred attributes characterizing the inferred instantiation parameters 434 and the inferred occurrence probabilities 444 may be determined based at least in part on a range of features whose values are determinable by particular kernels in a convolutional machine learning architecture. As a further example, the range of inferred attributes characterizing the inferred instantiation parameters 434 and the inferred occurrence probabilities 444 may be determined based at least in part on a range of features whose values are determinable by capsules that are characterized by squashing functions. Example CapsNet machine learning architectures are described in Sabour et al., “Dynamic Routing Between Capsules,” available at https://arxiv.org/abs/1710.09829.

At step/operation 405, the dimension-adjustment layers 414 of the categorical inference machine learning engine 111 generate a dimensionally-adjusted structured representation 435 of the categorical input data objects 431 based at least in part on the inferred instantiation parameters 434 and the inferred occurrence probabilities 444 determined in step/operation 404. In some embodiments, as generated by the subsequent capsule layers 413, the inferred instantiation parameters 434 and the inferred occurrence probabilities 444 may be in an initial structure that is not compatible with an expected input structure of the pre-merger FC layers 415 of the categorical inference machine learning engine 111. In some of those embodiments, the dimension-adjustment layers 414 are configured to transform the initial structure of the inferred instantiation parameters 434 and the inferred occurrence probabilities 444 to the expected input structure of the pre-merger FC layers 415. To do so, the dimension-adjustment layers 414 may use at least one of flattening operations, dimensionality reduction operations, etc. The dimension-adjustment layers 414 may further be configured to provide the dimensionally-adjusted structured representation 435 to the pre-merger FC layers 415 of the categorical inference machine learning engine 111.

For example, the initial structure of output data provided by the subsequent capsule layers 413 may correspond to a three-dimensional structure (e.g., a three-dimensional tensor) having a first dimension corresponding to the number of categorical input data objects 431 (i.e., number of input data samples), a second dimension corresponding to the number of inferred attributes, and a third dimension corresponding to a size of a vector that includes the inferred instantiation parameters 434 and the inferred occurrence probabilities 444 for each pair of an inferred attribute and a categorical input data object. Moreover, the expected input structure of the pre-merger FC layers 415 may correspond to a two-dimensional structure (e.g., a two-dimensional tensor). In the described example, to transform the initial structure of the inferred instantiation parameters 434 and the inferred occurrence probabilities 444 to the expected input structure of the pre-merger FC layers 415, the dimension-adjustment layers 414 may perform a flattening operation on the three-dimensional structure. For example, the dimension-adjustment layers 414 may convert the second and third dimensions of the three-dimensional structure into a new second dimension, e.g., where the second dimension includes, for each categorical input data object 431 corresponding to a row in the first dimension, a set of tuples generated based at least in part on a Cartesian product of the attribute set characterized by the second dimension and the vector value set in the third dimension values for the third row.

At step/operation 406, the pre-merger FC layers 415 of the categorical inference machine learning engine 111 are configured to process the dimensionally-adjusted structured representation 435 to generate a pre-merger latent representation 436 of the categorical input data objects 431. In some embodiments, to generate the pre-merger latent representation 436 of the categorical input data objects 431, the pre-merger FC layers 415 apply a set of trained parameters to the dimensionally-adjusted structured representation 435, e.g., applies a trained parameter to each value in the dimensionally-adjusted structured representation 435. In some embodiments, the pre-merger FC layers 415 include a group of feedforward FC neural network layers. The pre-merger FC layers 415 may provide the pre-merger latent representation 436 of the categorical input data objects 431 to numerical merger layers 416 of the categorical inference machine learning engine 111.

At step/operation 407, the numerical merger layers 416 of the categorical inference machine learning engine 111 merge the pre-merger latent representation 436 of the categorical input data objects 431 with numerical feature values 447 for the categorical input data objects 431 to generate a merged latent representation 437 of the categorical input data objects 431. A numeric feature value for a categorical input data object 431 may be a numeric value characterizing a numerically-defined property of the noted categorical input data object 431. For example, numeric feature values 447 characterizing a medical service event data object may include a patient age feature value for the corresponding medical service predictive entity, a patient weight value, a patient height value for the corresponding medical service predictive entity, a patient blood pressure value for the corresponding medical service predictive entity, a provider quality score value for the corresponding medical service predictive entity, etc.

The numerical merger layers 416 may be configured to process the pre-merger latent representation 436 of the categorical input data objects 431 along with the numerical feature values 447 for the categorical input data objects 431 in accordance with a set of trained parameters to merge the pre-merger latent representation 436 of the categorical input data objects 431 and the numerical feature values 447 and generate the merged latent representation 437 of the categorical input data objects 431. The numerical merger layers 416 may further be configured to provide the generated merged latent representation 437 to post-merger FC layers 417 of the categorical inference machine learning engine 111.

At step/operation 408, the post-merger FC layers 417 of the categorical inference machine learning engine 111 process the merged latent representation 437 of the categorical input data objects 431 to generate a final latent representation 438 of the categorical input data objects 431. In some embodiments, to generate the final latent representation 438 of the categorical input data objects 431, the post-merger FC layers 417 apply a set of trained parameters to the merged latent representation 437, e.g., apply a trained parameter to each value in the merged latent representation 437. In some embodiments, the post-merger FC layers 417 include a group of feedforward FC neural network layers. The post-merger FC layers 417 may provide the final latent representation 438 of the categorical input data objects 431 to final prediction layers 418 of the categorical inference machine learning engine 111.

At step/operation 409, the final prediction layers 418 of the categorical inference machine learning engine 111 process the final latent representation 438 of the categorical input data objects 431 to generate the predictions 451. In some embodiments, the final prediction layers 418 include layers of a Multi-Layered Perceptron (MLP) machine learning framework. In some embodiments, each categorical input data object 431 includes medical service information for a medical service event associated with the categorical input data object 431, and the predictions 451 for each categorical input data object 431 includes a predicted value (e.g., a predicted allowed insurance coverage value) for the medical service event associated with the categorical input data object.

In some embodiments, the final prediction layers 418 are further configured to determine, based at least in part on each predicted value for a categorical input data object of the categorical input data objects 431 (e.g., based at least in part on a measure of deviation of the predicted value from an actual initial value for the categorical data object), one or more claim audit need determinations (e.g., medical claim audit need determinations) and automatically perform one or more claim adjustments corresponding to the one or more claim adjustment need determinations. In some embodiments, the final prediction layers 418 are further configured to determine, based at least in part on each predicted value for a categorical input data object of the categorical input data objects 431 (e.g., based at least in part on a measure of deviation of the predicted value from an actual initial value for the categorical data object), one or more claim audit need determinations (e.g., medical claim audit need determinations) and automatically perform one or more claim adjustments corresponding to the one or more claim adjustment need determinations.

B. Regime-Based Categorical Predictive Inference

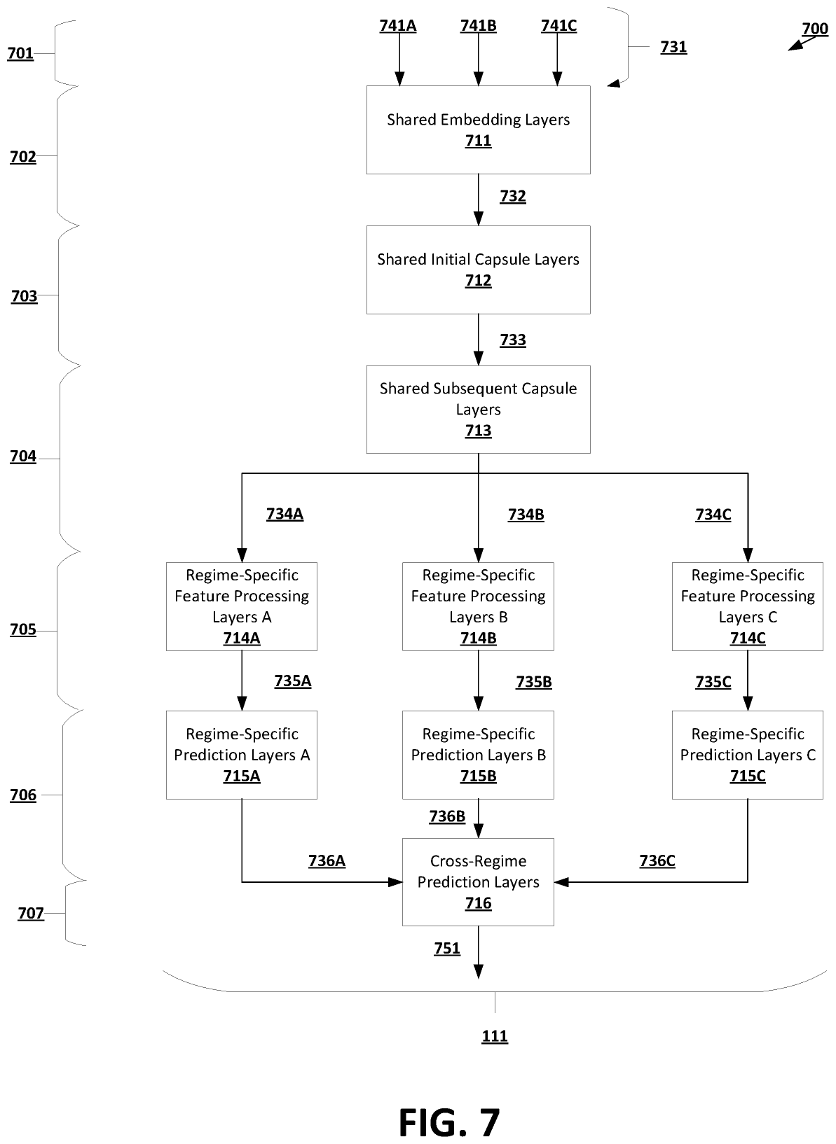

FIG. 7 is a data flow diagram of an example process 700 for performing a regime-based predictive inference based at least in part on categorical input data objects 731. Via the various steps/operations depicted in process 700, the categorical inference machine learning engine 111 can perform effective and efficient predictive inferences based at least in part on various regime-based streams of categorical input data objects 731 in order to generate reliable and effective predictions 751.

The process depicted in process 700 begins at step/operation 701 when the shared embedding layers 711 of the categorical inference machine learning engine 111 receive various categorical data streams 741A-C of the categorical input data objects 731. In some embodiments, a categorical input data object 731 is a data object that includes at least one categorical feature value, where a categorical feature value is a value that indicates association of the categorical input data object with a selected category of a plurality of discrete candidate categories. Each categorical input data object 731 may correspond to a predictive entity and include one or more categorical feature values, where each categorical feature value associated with a categorical input data object may in turn be associated with a categorical feature of one or more categorical features. An example of a categorical input data object is a medical service event data object that includes categorical information about a medical service event predictive entity (e.g., a medical visitation event predictive entity, a medical operation event predictive entity, a drug purchase event predictive entity, etc.). In some embodiments, the shared embedding layers 711 are configured to process various categorical data streams 741A-C using a shared set of machine learning layers, e.g., using a shared set of parameters. In some embodiments, step/operation 702 may be performed in accordance with the steps/operations depicted in FIG. 5 and described above with respect to step/operation 402 of process 400.

Examples of categorical feature values for a medical service event data object may include location-identifying categorical feature values for a medical service predictive entity, medical-procedure-code-based categorical feature values for a medical service predictive entity, medical-diagnosis-code-based categorical feature values (e.g., medical-diagnosis-code-based categorical feature values characterized by a medical diagnoses classification system such as the DRG system) for a medical service predictive entity, point-of-service-related categorical feature values for a medical service predictive entity, etc. In some embodiments, the categorical input data objects 731 are each associated with a value indicator, where the value indicator for a categorical input data object 731 may be an initial indicator of a real-world value of the predictive entity corresponding to the categorical input data object 731. For example, a value indicator for a medical service event data object may be determined based at least in part on an actual value charged by a medical provider for the medical service event predictive entity that corresponds to the medical service event data object.

In some embodiments, the categorical input data objects 731 are divided into n value regime designations based at least in part on the value indicators for the categorical input data objects 731, where a value regime designation corresponds to one or more subranges of a total range of the value indicators, and where n may be a value that is greater than or equal to two and may be determined based at least in part on a hyper-parameter of the categorical inference machine learning engine 111. For example, the categorical input data objects 731 may be divided into three value regime designations, where a first value regime designation may include categorical input data objects 731 whose respective value indicators fall within a first standard deviation of a mean of a distribution of all the value indicators for the categorical input data objects 731, a second value regime designation may include categorical input data objects 731 whose respective value indicators fall between the first standard deviation and a second standard deviation of the mean of the distribution of all the value indicators for the categorical input data objects 731, and a third value regime designation may include categorical input data objects 731 whose respective value indicators fall outside the second standard deviation. As another example, the categorical input data objects 731 may be divided into three value regime designations, where a first value regime designation may include categorical input data objects 731 whose respective value indicators are below a first threshold (e.g., below 200 hundred dollars), a second value regime designation may include categorical input data objects 731 whose respective value indicators are between the first threshold and a second threshold (e.g., between 200 hundred dollars and 500 dollars), and a third value regime designation may include categorical input data objects whose respective value indicators are above the second threshold (e.g., above 500 dollars).

In some embodiments, each categorical data stream 741A-C is associated with a value regime designation and includes at least a portion of the categorical data associated with the categorical input data objects 731 having the corresponding value regime designation. For example, in the example categorical inference machine learning engine 111 depicted in FIG. 7, the categorical data stream 741A may be associated with a low value regime designation and thus include categorical data associated with the low value regime designation, the categorical data stream 741B may be associated with a medium value regime designation and thus include categorical data associated with the medium value regime designation, and the categorical data stream 741C may be associated with a high value regime designation and thus include categorical data associated with the high value regime designation. While the exemplary process 700 of FIG. 7 depicts three categorical data streams, a person of ordinary skill in the relevant technology will recognize that any number of categorical data streams may be modeled and provided without deviating from the spirit of the regime-based categorical inference aspects of the present invention.

At step/operation 702, the shared embedding layers 711 of the categorical inference machine learning engine 111 process the categorical input data objects 731 to generate one or more embedded feature representations 732 for each categorical input data object 731 and provide the generated embedded feature representations 732 to shared initial capsule layers 712 of the categorical inference machine learning engine 111. In some embodiments, an embedded feature representation is a mapping of one or more categorical feature values to an n-dimensional space, where each feature dimension of the n feature dimensions may be characterized by a numeric range and where the dimension count n may be defined by one or more hyper-parameters of the categorical inference machine learning engine 111. In some embodiments, an embedded feature representation is a mapping of a numerical token (e.g., an integer token) generated based at least in part on one or more categorical features value to an n-dimensional space, where each feature dimension of the n feature dimensions may be characterized by a numeric range and where the dimension count n may be defined by one or more hyper-parameters of the categorical inference machine learning engine 111.

In some embodiments, to generate an embedded feature representation 732 based at least in part on a categorical feature value associated with a categorical input data object 731, the shared embedding layers 711 first tokenize the categorical feature value as an integer and then maps the tokenized categorical feature value to an n-dimensional space based at least in part on a look-up table, where at least some of the parameters defining the look-up table may be learned through at least one training procedure. In general, any combination of one or more embedding techniques can be utilized to convert at least one categorical feature value into a corresponding embedded feature representation 732. In some embodiments, the shared embedding layers 711 are configured to map categorical feature values associated with various distinct categorical features into embedded feature representations 732 of the same length and the same structure, e.g., vectors of length n where each value of the vector represents the same ordered set of embedded features across the various categorical feature values. In some embodiments, each embedded feature representation 732 has a shared embedding structure relative to the other embedded feature representations 732. In some embodiments, the embedding layers 411 are configured to map categorical feature values associated with distinct categorical features into embedded feature representations 732 having feature-specific representations.

At step/operation 703, the shared initial capsule layers 712 of the categorical inference machine learning engine 111 process the embedded feature representations 732 to generate one or more instantiation parameters 733 for each embedded feature representation 732. In some embodiments, an initial instantiation parameter 733 for a corresponding embedded feature representation 732 that is in turn associated with a corresponding categorical input data object 731 describes an extracted occurrence property of the corresponding embedded feature representation 732 with respect to the corresponding embedded feature representation 732. For example, a particular initial instantiation parameter 733 may describe an orientation of the corresponding embedded feature representation 732 within a spatial space generated based at least in part on the corresponding categorical input data object 731. As another example, a particular initial instantiation parameter 733 may describe an intensity of occurrence of the corresponding embedded feature representation 732 with respect to the corresponding categorical input data object 731. As a further example, a particular initial instantiation parameter 733 may describe a predictive significance of the corresponding embedded feature representation 732 to making particular predictive inferences.

In some embodiments, the shared initial capsule layers 712 further generate an initial occurrence probability for an embedded feature representation. In some embodiments, an initial occurrence probability for a corresponding embedded feature representation 732 that is in turn associated with a corresponding categorical input data object 731 describes a probability of occurrence of the corresponding embedded feature representation 732 with respect to the corresponding categorical input data object 431. For example, a particular initial occurrence probability may describe a likelihood that the corresponding embedded feature representation 732 describes a property of the corresponding categorical input data object 731.

In some embodiments, the shared initial capsule layers 712 are configured to process various categorical data streams 741A-C of using a shared set of machine learning layers, e.g., using a shared set of parameters. In some embodiments, step/operation 703 may be performed in accordance with the steps/operations depicted in FIG. 6 and described above with respect to step/operation 403 of process 400. The shared initial capsule layers 712 may provide the initial instantiation parameters 434 and/or the initial occurrence probabilities to subsequent capsule layers 703 of the categorical inference machine learning engine 111.

At step/operation 704, the shared subsequent capsule layers 713 of the categorical inference machine learning engine 111 process the initial instantiation parameters 733 for the embedded feature representations 732 (and optionally the initial feature probabilities for the embedded feature representations 732) to generate a regime-specific capsule output stream 734A-C for each categorical feature stream 741A-C. In some embodiments, the regime-specific capsule output stream 734A-C for a categorical feature stream 741A-C may include, for each categorical data object 731 associated with the particular categorical feature stream 741A-C, one or more inferred instantiation parameters for the categorical input data object 731 and one or more inferred occurrence probabilities for the categorical input data object 731. An inferred instantiation parameter 734 for a categorical input data object 731 may describe an inferred occurrence property of a corresponding inferred attribute with respect to the particular categorical input data object 731. An inferred occurrence probability 744 for a categorical input data object 731 may describe a predicted probability of occurrence of a corresponding inferred attribute with respect to the categorical input data object 731. In some embodiments, the shared subsequent capsule layers 713 are configured to process various categorical data streams 741A-C using a shared set of machine learning layers, e.g., using a shared set of parameters. The subsequent capsule layers 713 may provide the inferred instantiation parameters 734 and/or the inferred occurrence probabilities 444 to regime-specific feature processing layers 714A-C of the categorical inference machine learning engine 111.

In some embodiments, the range of inferred attributes characterizing the inferred instantiation parameters 734 and the inferred occurrence probabilities 744 may be determined based at least in part on a range of features whose values are determinable by particular capsules in a CapsNet machine learning architecture. For example, the range of inferred attributes characterizing the inferred instantiation parameters 434 and the inferred occurrence probabilities 444 may be determined based at least in part on a range of features whose values are determinable by particular capsules in a primary capsule layer of a CapsNet machine learning architecture. As another example, the range of inferred attributes characterizing the inferred instantiation parameters 734 and the inferred occurrence probabilities 744 may be determined based at least in part on a range of features whose values are determinable by particular kernels in a convolutional machine learning architecture. As a further example, the range of inferred attributes characterizing the inferred instantiation parameters 734 and the inferred occurrence probabilities 744 may be determined based at least in part on a range of features whose values are determinable by capsules that are characterized by squashing functions. Example CapsNet machine learning architectures are described in Sabour et al., “Dynamic Routing Between Capsules,” available at https://arxiv.org/abs/1710.09829.

At step/operation 705, the regime-specific feature processing layers 714A-C of the categorical inference machine learning engine 111 process the regime-specific capsule output streams 434A-C received from the shared subsequent machine learning layers 713 to generate regime-specific latent representation 735A-735C for each categorical feature stream 741A-C. In some embodiments, each of the regime-specific feature processing layers 714A-C is configured to process a structured representation of the inferred instantiation parameters 734 and the inferred occurrence probabilities 744 associated with a corresponding value regime designation in order to generate a corresponding regime-specific latent representation 735A-C for the corresponding value regime designation.

For example, as depicted in FIG. 7, the regime-specific feature processing layer a 714A is configure to process the structured representation associated with a first value regime designation to generate a corresponding regime-specific latent representation A 735A, the regime-specific feature processing layer B 714B is configure to process the structured representation associated with a second value regime designation to generate a corresponding regime-specific latent representation B 735B, and the regime-specific feature processing layer C 714C is configure to process the structured representation associated with a third value regime designation to generate a corresponding regime-specific latent representation C 735C. The regime-specific feature processing layers 714A-C may further be configured to provide the generated regime-specific latent representation 735A-735C to regime-specific prediction layers 715A-C of the categorical inference machine learning engine 111.

At step/operation 706, each regime-specific prediction layer 715A-C of the categorical inference machine learning engine 111 receives a regime-specific latent representation 735A-C from a corresponding regime-specific feature processing layer 714A-C and processes the received regime-specific latent representation 735A-C to generate regime-specific predictions 736A-C for a corresponding value regime designation that is associated with corresponding regime-specific feature processing layer 714A-C. For example, in the exemplary categorical inference machine learning engine 111 depicted in FIG. 7, the regime-specific prediction layer A 715A is configured to process the regime-specific latent representation A 735A received from the corresponding regime-specific feature processing layer A 715A in order to generate a regime-specific prediction 736A for a corresponding first value regime designation, the regime-specific prediction layer B 715B is configured to process the regime-specific latent representation B 735B received from the corresponding regime-specific feature processing layer B 715B in order to generate a regime-specific prediction 736B for a corresponding second value regime designation, and the regime-specific prediction layer C 715C is configured to process the regime-specific latent representation C 735C received from the corresponding regime-specific feature processing layer C 715C in order to generate a regime-specific prediction 736C for a corresponding third value regime designation. In some embodiments, at least one regime-specific prediction layer 715A-C includes one or more final prediction layers, such as one or more MLP layers.

At step/operation 707, the cross-regime prediction layers 716 receive the regime-specific latent representations 735A-C from the regime-specific prediction layer 715A-C and processes the regime-specific latent representations 735A-C to generate the predictions 751. In some embodiments, each categorical input data object 731 includes medical service information for a medical service event associated with the categorical input data object 731, and the predictions 751 for each categorical input data object 731 includes a predicted value (e.g., a predicted allowed insurance coverage value) for the medical service event associated with the categorical input data object.

In some embodiments, the cross-regime prediction layers 716 are further configured to determine, based at least in part on each predicted value for a categorical input data object of the categorical input data objects 731, one or more claim audit need determinations (e.g., medical claim audit need determinations) and automatically perform one or more claim adjustments corresponding to the one or more claim adjustment need determinations. In some embodiments, the cross-regime prediction layers 716 are further configured to determine, based at least in part on each predicted value for a categorical input data object of the categorical input data objects 731, one or more claim audit need determinations (e.g., medical claim audit need determinations) and automatically perform one or more claim adjustments corresponding to the one or more claim adjustment need determinations.

C. Training a Categorical Inference Machine Learning Engine

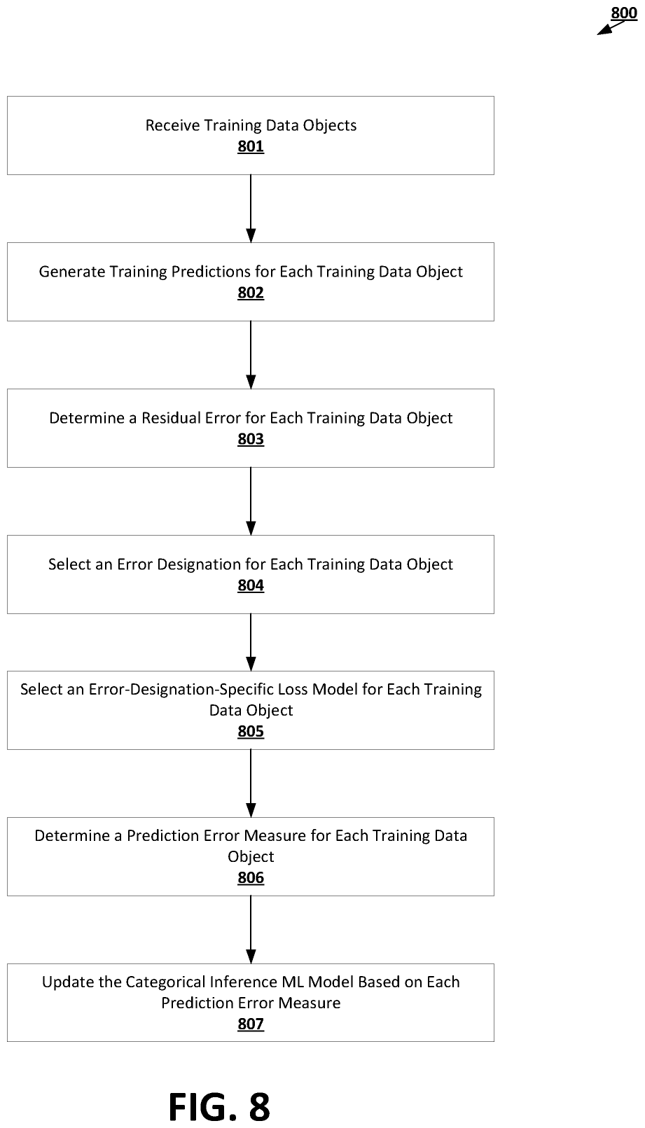

FIG. 8 is a flowchart diagram of an example process 800 for training the categorical inference machine learning engine 111 to perform predictive inference based at least in part on categorical training input data. Via the various steps/operations of the process 800, the training engine 112 of the categorical inference computing entity 106 can efficiently and effectively train at least one of a general categorical inference machine learning engine (e.g., a general categorical inference machine learning engine having the structure depicted in FIG. 4) and a regime-specific categorical inference machine learning engine (e.g., a regime-specific categorical inference machine learning engine having the structure depicted in FIG. 7).

At step/operation 801, the training engine 112 receives one or more training data objects, where each training data object is associated with one or more training categorical feature values and one or more ground-truth predictions. A ground-truth may be a value that indicates a real-world observation about a desirable value of a desired property of a predictive entity associated with a corresponding training data object. For example, when the training data object is a medical service event data object, the ground-truth predictions for the medical service event data object may include a financial value estimation for the corresponding medical service event predictive entity as determined by an expert evaluator such as a medical practitioner and/or as determined by an auditor.

At step/operation 802, the training engine 112 processes the training categorical feature values associated with a training data object of the one or more training data objects using the categorical inference machine learning engine 111 in order to generate one or more training predictions for the particular training data object. In some embodiments, the categorical inference machine learning engine 111 may include at least one of a general categorical inference machine learning engine (e.g., a general categorical inference machine learning engine having the structure depicted in FIG. 4) and a regime-specific categorical inference machine learning engine (e.g., a regime-specific categorical inference machine learning engine having the structure depicted in FIG. 7). Although the exemplary process 800 is described with respect to a machine learning engine configured to process categorical input data, a person of ordinary skill in the relevant technology will recognize that the disclosed techniques can be used to train any kind of an machine learning model configured to process and perform predictions using any kind of input data.

At step/operation 803, the training engine 112 determines a residual error for each training data object based at least in part on a measure of difference between the training predictions for the training data object and the ground-truth predictions for training data object. In some embodiments, the residual error measure may be calculated based at least in part on a ratio of an absolute value of a measure of difference between a training value prediction for the corresponding training data object and a ground-truth value prediction for the training data object and the ground-truth value prediction for the training data object (i.e., based at least in part on |training value prediction−ground-truth prediction|/ground-truth prediction).

At step/operation 804, the training engine 112 selects an error designation for each training data object based at least in part on the residual error for the training data object. In some of those embodiments, the training engine 112 divides the training data objects into m error designations based at least in part on the residual errors for the training data objects, where m may be determined based at least in part on a hyper-parameter of the training engine 112. For example, the training engine 112 may divide the training data objects into three error designations, where the first error designation may include training data objects whose residual error falls below a first threshold (e.g., δ), the second error designation may include training data objects whose residual error falls between the first threshold and a second threshold (e.g., n*δ), and the third error designation may include training data objects whose residual error falls above the second threshold. At least some of the values used to determine the error designation thresholds (e.g., the values n and 6 in the described example) may be determined based at least in part on a distribution of residual errors across various training data objects, based at least in part on one or more training procedures, and/or based at least in part on one or more hyper-parameters of the training engine 112.

At step/operation 805, the training engine 112 selects an error-designation-specific loss model for each training data object based at least in part on the selected error designation for the training data object. In some embodiments, each error designation is associated with an error-designation-specific loss model. For example, in some embodiments, the error designations include a low error designation, a medium error designation, and a high error designation. In some of those embodiments, the error-designation-specific loss models include a high-outlier-resistant loss model for the low error designation, a medial-outlier-resistant loss model for the medium error designation, and a low-outlier-resistant loss model for the high error designation.

In some embodiments, the high-outlier-resistant loss model is a loss model that has a lower level of tolerance for outlier predictions compared to the medial-outlier-resistant loss model and the low-outlier-resistant loss model. An example of a high-outlier-resistant loss model is a squared-error-based loss model, such as the loss model described by the equation ½(y−f(x))², if|y−f(x)|≤δ, where y is a ground-truth prediction for a particular training data object, f(x) is a training prediction for the particular training data object, and δ is a first error designation threshold.

In some embodiments, a medial-outlier-resistant loss model is a loss model that has a level of tolerance for outlier prediction that is higher than the high-outlier-resistant loss model and lower than the low-outlier-resistant loss model. An example of a medial-outlier-resistant loss model is a Huber loss model or a modified Huber loss model, such as the loss model given by the equation ½δ|y−f(x)|+¼δ², if δ≤|y−f(x)|≤nδ, where y is a ground-truth prediction for a particular training data object, f(x) is a training prediction for the particular training data object, δ is a first error designation threshold, and nδ is a second error designation threshold.

In some embodiments, a low-outlier-resistant loss model is a loss model that has a level of tolerance for outlier prediction that is lower than the high-outlier-resistant loss model and the medial-outlier-resistant loss model. An example of a low-outlier-resistant loss model is a Cauchy loss model or a modified Cauchy loss model, such as the loss model given by the equation

$$\frac{1}{4} ( 1 + 2 n ) {δ}^{2} + log ( 1 + \frac{\vert y - f ( x ) \vert}{2 n δ} ) ,$$

where y is a ground-truth prediction for a particular training data object, f(x) is a training prediction for the particular training data object, δ is a first error designation threshold, and nδ is a second error designation threshold.

In some embodiments, the training engine 112 is associated with a hybrid loss model, where the hybrid loss model designates different loss models for different residual error designations associated with predictions by a categorical inference machine learning engine 111. For example, the training engine 112 may be associated with a hybrid loss model defined by the below equation, where y is a ground-truth prediction for a particular training data object, f(x) is a training prediction for the particular training data object, δ is a first error designation threshold, and nδ is a second error designation threshold.

$$\{ \begin{matrix}\frac{1}{2} {( y - f ( x ) )}^{2} , if \vert y - f ( x ) \vert \leq δ \\ \frac{1}{2} δ \vert y - f ( x ) \vert + \frac{1}{4} {δ}^{2} , if δ \leq for \vert y - f ( x ) \vert \leq n δ \\ \frac{1}{4} ( 1 + 2 n ) {δ}^{2} + log ( 1 + \frac{\vert y - f ( x ) \vert}{2 n δ} ) , otherwise\end{matrix}$$

At step/operation 805, the training engine 112 determines a prediction error measure for each training data object of the one or more training data objects using the error-designation-specific loss model for the training data object. In some embodiments, the training engine 112 applies the output of the error-designation-specific loss model for a training data object as the prediction error measure for the training data object. For example, given a training data object classified as having a low residual error designation, the training engine 112 may supply a high-outlier-resistant loss model with values corresponding to the training data object to generate the prediction error measure for the training data object.

At step/operation 806, the training engine 112 updates the categorical inference machine learning engine 111 based at least in part on each prediction error measure for a training data object of the one or more training data objects. In some embodiments, to update the categorical inference machine learning engine 111 based at least in part on each prediction error measure for a training data object of the one or more training data objects, the training engine 112 utilizes an optimization algorithm such as gradient descent. In some embodiments, to update a multi-layered categorical inference machine learning engine 111 based at least in part on each prediction error measure for a training data object of the one or more training data objects, the training engine 112 utilizes a backpropogation algorithm. In some embodiments, to update a multi-layered categorical inference machine learning engine 111 based at least in part on each prediction error measure for a training data object of the one or more training data objects, the training engine 112 utilizes an end-to-end training algorithm.

### V. CONCLUSION

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A computer-implemented method for performing predictive inference using a categorical inference machine learning engine and based at least in part on categorical input data, the computer-implemented method comprising:
receiving one or more categorical input data objects, wherein each of the categorical input data objects is associated with one or more categorical feature values;
generating, using one or more embedding layers of the categorical inference machine learning engine and based at least in part on each of the categorical input data objects, one or more embedded feature representations for the corresponding categorical input data object;
for each embedded feature representation associated with the corresponding categorical input data object, generating, using one or more initial capsule layers of the categorical inference machine learning engine and based at least in part on the corresponding embedded feature representation, one or more initial instantiation parameters indicating an extracted occurrence property of the corresponding embedded feature representation with respect to the corresponding categorical input data object;
generating, using one or more subsequent capsule layers and based at least in part on each initial instantiation parameter, one or more inferred instantiation parameters for the corresponding categorical input data object, wherein each inferred instantiation parameter for the corresponding categorical input data object indicates an inferred occurrence property of a corresponding inferred attribute with respect to the corresponding categorical input data object; and
generating one or more predictions based at least in part on each of the one or more inferred instantiation parameters.

2. The computer-implemented method of claim 1, wherein:
the one or more initial capsule layers comprise a plurality of spatial fully-connected layers and one or more localized convolution layers,
the plurality of spatial fully-connected layers are configured to process each embedded feature representation based at least in part on a spatial relationship between the embedded feature representation and the corresponding categorical data object to generate a spatial feature representation for the embedded feature representation, and
the one or more localized convolution layers are configured to process each spatial feature representation for an embedded feature representation in accordance with one or more feature extraction kernels to generate each of the one or more initial instantiation parameters for the embedded feature representation.

3. The computer-implemented method of claim 2, wherein the plurality of spatial fully-connected layers are wrapped by a time-distributed layer.

4. The computer-implemented method of claim 1, wherein the one or more initial capsule layers are further configured to generate, for each embedded feature representation associated with the corresponding categorical input data object, an initial occurrence probability for the corresponding embedded feature representation with respect to the corresponding embedded categorical input data object.

5. The computer-implemented method of claim 1, wherein the one or more updated capsule layers are further configured to generate an inferred probability for each corresponding inferred attribute with respect to the corresponding categorical input data object.

6. The computer-implemented method of claim 1, wherein generating the one or more predictions based at least in part on each of the one or more inferred instantiation parameters for the categorical input data object comprises:
generating, by one or more dimension-adjustment layers of the categorical inference machine learning engine, a dimensionally-adjusted structured representation of the one or more categorical input data objects based at least in part on each of the one or more inferred instantiation parameters for a categorical input data object;
processing, by one or more per-merger fully-connected layers of the categorical inference machine learning engine, the dimensionally-adjusted structured representation to generate a pre-merger latent representation of the one or more categorical input data objects;
processing, by one or more numerical merger layers of the categorical inference machine learning engine and based at least in part on each of the one or more numerical feature values for a categorical input data object of the one or more categorical input data objects, the pre-merger latent representation to generate a merged latent representation of the one or more categorical input data objects;
processing, by one or more post-merger fully-connected layers of the categorical inference machine learning engine, the pre-merger latent representation to generate a final latent representation of the one or more categorical input data objects; and
processing, by one or more final prediction layers of the one or more categorical input data objects, the final latent representation to generate the one or more predictions.

7. The computer-implemented method of claim 1, wherein generating the one or more predictions based at least in part on each of the one or more inferred instantiation parameters for a categorical input data object of the one or more categorical input data objects comprises:
identifying a value regime designation of a plurality of regime designation values for each categorical input data object based at least in part on a respective value indicator for the categorical input data object;
receiving a regime-specific dimensionally-adjusted structured representation associated with each particular value regime designation of the plurality of regime designation values based at least in part on each of the one or more instantiation parameters associated with a categorical input data object which is in turn associated with the particular value regime;
processing each regime-specific dimensionally-adjusted structured representation for a particular value regime designation of the plurality of value regime designations by one or more regime-specific feature processing layers for the particular value regime designation to generate one or more regime-specific latent representations; and
generating the one or more predictions based at least in part on each of the one or more regime-specific prediction outputs for a value regime designation of the plurality of value regime designations.

8. The computer-implemented method of claim 1, wherein training the categorical inference machine learning engine comprises:
receiving one or more training data objects, wherein each training data object is associated with one or more training categorical feature values and one or more ground-truth predictions;
processing each of the one or more training categorical feature values associated with a training data object using the categorical inference machine learning engine to generate one or more training predictions for the particular training data object;
determining a residual error measure for each training data object based at least in part on the one or more ground-truth predictions for the training data object and the one or more training predictions for the training data object;
selecting an error designation of a plurality of error designations for each training data object based at least in part on the residual error measure for the training data object;
selecting an error-designation-specific loss model of a plurality of error-designation-specific loss models for each training data object based at least in part on the error designation for the training data object;
determining a prediction error measure for each training data object using the error-designation-specific loss model for the training data object; and
updating the categorical inference machine learning engine based at least in part on each prediction error measure for a training data object.

9. The computer-implemented method of claim 8, wherein:
the plurality of error designations comprises a low error designation, a medium error designation, and a high error designation; and
the plurality of error-designation-specific loss models comprises a high-outlier-resistant loss model for the low error designation, a medial-outlier-resistant loss model for the medium error designation, and a low-outlier-resistant loss model for the high error designation.

10. The computer-implemented method of claim 9, wherein the high-outlier-resistant loss model is determined based at least in part on a squared-error-based loss model.

11. The computer-implemented method of claim 9, wherein the medial-outlier-resistant loss model is determined based at least in part on an absolute-deviation-based loss model.

12. The computer-implemented method of claim 9, wherein the medial-outlier-resistant loss model is determined based at least in part on a Huber loss model.

13. The computer-implemented method of claim 9, wherein the low-outlier-resistant loss model is determined based at least in part on a Cauchy loss function.

14. The computer-implemented method of claim 1, wherein each embedded feature representation has a shared embedding structure relative to other embedded feature representations of the one or more embedded feature representations.

15. The computer-implemented method of claim 1, wherein:
each categorical input data object of the one or more categorical input data objects comprises medical service information for a medical service event associated with the categorical input data object, and
the one or more predictions for a categorical input data object of the one or more categorical input data objects comprise a predicted value for the medical service event associated with the categorical input data object.

16. The computer-implemented method of claim 15, further comprising:
determining, based at least in part on each predicted value for a categorical input data object of the one or more categorical input data objects, one or more claim adjustment need determinations; and
automatically performing one or more claim adjustments corresponding to the one or more claim adjustment need determinations.

17. The computer-implemented method of claim 15, further comprising:
determining, based at least in part on each predicted value for a categorical input data object of the one or more categorical input data objects, one or more claim audit need determinations; and
automatically performing the one or more claim audit corresponding to the one or more claim audit need determinations.

18. An apparatus for performing predictive inference using a categorical inference machine learning engine and based at least in part on categorical input data, the apparatus comprising at least one processor and at least one memory including program code, the at least one memory and the program code configured to, with the processor, cause the apparatus to at least:
receive one or more categorical input data objects, wherein each of the categorical input data objects is associated with one or more categorical feature values;
generate, using one or more embedding layers of the categorical inference machine learning engine and based at least in part on each of the categorical input data objects, one or more embedded feature representations for the corresponding categorical input data object;
for each embedded feature representation associated with the corresponding categorical input data object, generate, using one or more initial capsule layers of the categorical inference machine learning engine and based at least in part on the corresponding embedded feature representation, one or more initial instantiation parameters indicating an extracted occurrence property of the corresponding embedded feature representation with respect to the corresponding categorical input data object;
generate, using one or more subsequent capsule layers and based at least in part on each initial instantiation parameter, one or more inferred instantiation parameters for the corresponding categorical input data object, wherein each inferred instantiation parameter for the corresponding categorical input data object indicates an inferred occurrence property of a corresponding inferred attribute with respect to the corresponding categorical input data object; and
generate one or more predictions based at least in part on each of the one or more inferred instantiation parameters.

19. The apparatus of claim 18, wherein:
the one or more initial capsule layers comprise a plurality of spatial fully-connected layers and one or more localized convolution layers,
the plurality of spatial fully-connected layers are configured to process each embedded feature representation based at least in part on a spatial relationship between the embedded feature representation and the corresponding categorical data object to generate a spatial feature representation for the embedded feature representation, and
the one or more localized convolution layers are configured to process each spatial feature representation for an embedded feature representation in accordance with one or more feature extraction kernels to generate each of the one or more initial instantiation parameters for the embedded feature representation.

20. A computer program product for performing predictive inference using a categorical inference machine learning engine and based at least in part on categorical input data, the computer program product comprising at least one non-transitory computer-readable storage medium having computer-readable program code portions stored therein, the computer-readable program code portions configured to:
receive one or more categorical input data objects, wherein each of the categorical input data objects is associated with one or more categorical feature values;
generate, using one or more embedding layers of the categorical inference machine learning engine and based at least in part on each of the categorical input data objects, one or more embedded feature representations for the corresponding categorical input data object;
for each embedded feature representation associated with the corresponding categorical input data object, generate, using one or more initial capsule layers of the categorical inference machine learning engine and based at least in part on the corresponding embedded feature representation, one or more initial instantiation parameters indicating an extracted occurrence property of the corresponding embedded feature representation with respect to the corresponding categorical input data object;
generate, using one or more subsequent capsule layers and based at least in part on each initial instantiation parameter, one or more inferred instantiation parameters for the corresponding categorical input data object, wherein each inferred instantiation parameter for the corresponding categorical input data object indicates an inferred occurrence property of a corresponding inferred attribute with respect to the corresponding categorical input data object; and
generate one or more predictions based at least in part on each of the one or more inferred instantiation parameters.
